(19) United States
(12) Patent Application Publication
(10) Pub. No.: US 2025/0213383 A1
(43) Pub. Date: Jul. 3, 2025

(54) ANTI-BLOCKAGE URINE BAG

(71) Applicants: CHIEN-CHUNG SU, Taichung City (TW); YI-CHIA CHANG, Taichung City (TW); YA-WEN KU, Taichung City (TW); ARCHER BIOTECHNOLOGY DEVELOPMENT CO., LTD., Taichung City (TW)

(72) Inventors: CHIEN-CHUNG SU, Taichung City (TW); YI-CHIA CHANG, Taichung City (TW); YA-WEN KU, Taichung City (TW); PING-I HSU, Kaohsiung City (TW)

(21) Appl. No.: 18/402,089

(22) Filed: Jan. 2, 2024

(51) Int. Cl. A61F 5/44

(52) U.S. Cl. CPC A61F 5/4404

(57) ABSTRACT

An anti-blockage urine bag includes a urine bag body with a passing hole, a urethral catheter with an end connected to a user's urinary tract and another end having a passing hole for passing the urine bag body, and an anti-folding tube having a flow hole, a plug-in part and a hollow support part. The urethral catheter is communicated to the flow hole and sheathed on the plug-in part, the plug-in part keeps the urethral catheter straight, the hollow support part is contained in the urine bag body and has an external diameter greater than the external diameter of the plug-in part, and the hollow support part can hold up the interior of the urine bag body. In this way, the anti-folding tube can prevent the urethral catheter and urine bag body from being bent which may cause blockage of urine, so as to ensure a smooth normal catheterization.

### BACKGROUND OF THE DISCLOSURE

### Technical Field

The present disclosure relates to a urine bag structure, more particularly relates to an anti-blockage urine bag.

### Description of the Related Art

Patients (such as those having or recently had a surgery) or disabled people who are unable to urinate on their own usually need to use a urine bag. With reference to FIGS. 7 and 8 for a conventional urine bag structure, the conventional urine bag structure generally includes a urine bag body 91, a urethral catheter 92 and a hanger 93. Two fixing holes 911 are formed at two corners of the upper side of the urine bag body 91 respectively, the urethral catheter 92 has a first connection part 921 and a second connection part 922, the first connection part 921 is provided for connecting to a user's urinary tract, the second connection part 922 is passed and communicated to the urine bag body 91, the middle of the lower side of the hanger 93 is provided with a through hole 931, and two sides of the hanger 93 are extended downwardly to form two hook parts 932 and upwardly to form two hooks 933.

During use, the through hole 931 of the hanger 93 is used for passing the urethral catheter 92, the two hook parts 932 of the hanger 93 are hooked into the two fixing holes 911 of the urine bag body 91, and the two hooks 933 of the hanger 93 are hooked to the side of a bag or any other appropriate position, so that the user's urine will flow through the urethral catheter 92 into the urine bag body 91 for collection.

However, when the conventional urine bag structure is in use, the urine bag body 91 is often deformed due to the weight of the urine received as shown in FIG. 9, which will result in bending at the position of the urethral catheter 92 adjacent to the urine bag body 91, and the affixation of the second connection part 922 of the urethral catheter 92 to the inner wall of the urine bag body 91, so as to block the urine and affect the normal smooth catheterization. Furthermore, the conventional urine bag has the disadvantages of complicated structure, inconvenient assembly, high manufacturing cost, and low cost-effectiveness.

### SUMMARY OF THE DISCLOSURE

The present disclosure provides an anti-blockage urine bag, including: a urine bag body, with a passing hole formed on the upper side of the urine bag body and communicated to the interior of the urine bag body: A urethral catheter, connected and communicated to the urine bag body, and having a first connection part and a second connection part configured to be opposite to each other, the first connection part being provided for connecting to a user's urinary tract, the second connection part being passed into the passing hole of the urine bag body and disposed inside the urine bag body; and an anti-folding tube, communicated between the urine bag body and the urethral catheter, and having a plug-in part and a hollow support part, a flow hole being formed and penetrating between the plug-in part and the hollow support part, wherein the second connection part of the urethral catheter is sheathed on the plug-in part, the flow hole is communicated to the urethral catheter, the plug-in part keeps the urethral catheter straight: the hollow support part is contained in the urine bag body, the hollow support part has an external diameter greater than the external diameter of the plug-in part, and the hollow support part is capable of holding up the interior of the urine bag body.

In the structure above, the present disclosure provides the anti-folding tube between the urethral catheter and the urine bag body, the plug-in part of the anti-folding tube can keep the urethral catheter straight, and the hollow support part of the anti-folding tube can hold up the urine bag body, thereby avoiding the blockage of urine to ensure a normal smooth catheterization, and effectively improving the conventional urine bag which is folded easily and will cause the blockage of urine frequently.

In addition, the present disclosure has the advantages of simple structure, easy assembly, low manufacturing cost and high cost-effectiveness.

### BRIEF DESCRIPTION OF THE DRAWINGS

FIG. 1 is a perspective view of the present disclosure;

FIG. 2 is an exploded view of the present disclosure:

FIG. 3 is a cross-sectional view of the present disclosure:

FIG. 4 is an enlarged view of Part A1 of FIG. 3:

FIG. 5 is a schematic view of a using status of the present disclosure:

FIG. 6 is an enlarged view of Part A2 of FIG. 5:

FIG. 7 is a perspective view of a conventional urine bag:

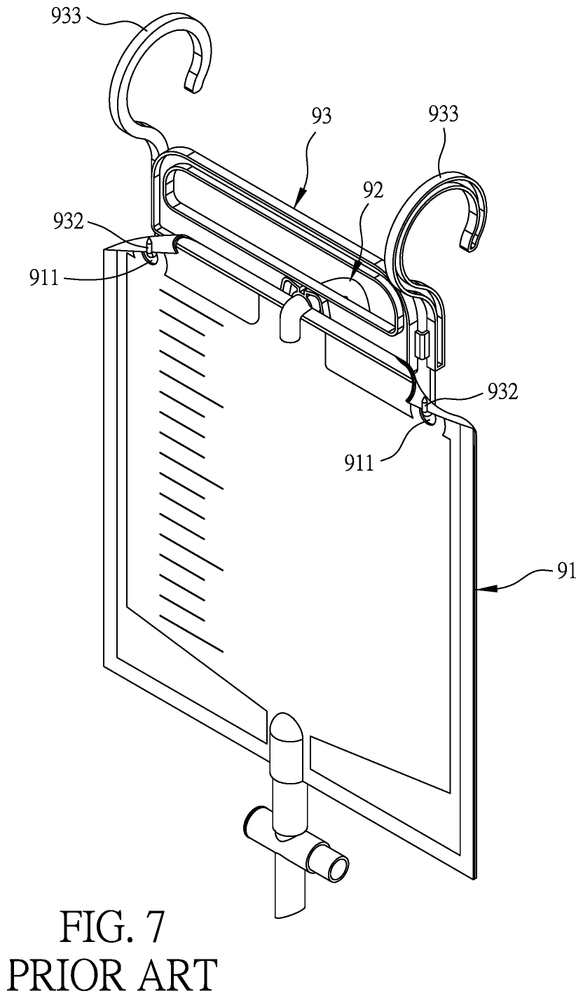

FIG. 8 is an exploded view of a conventional urine bag; and

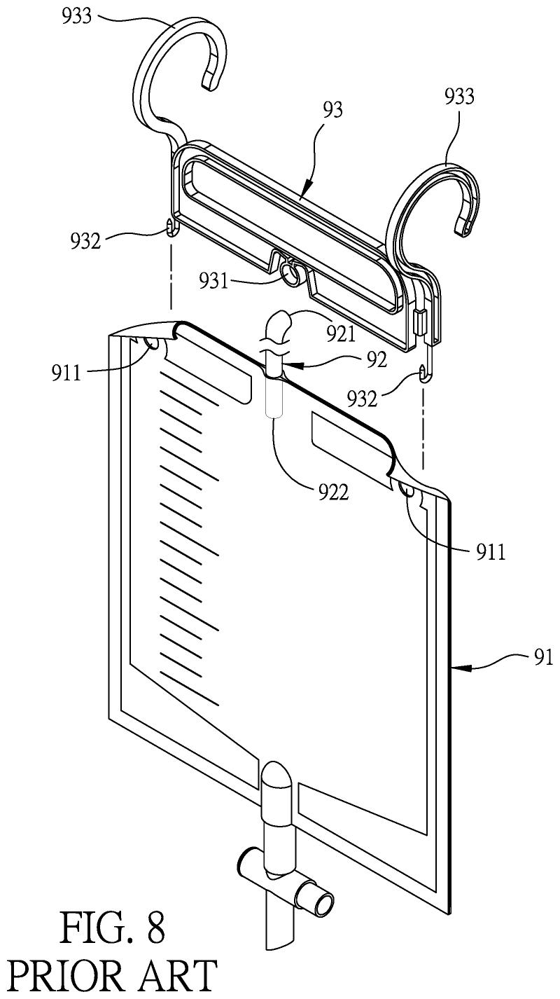

FIG. 9 is a schematic view of a using status of a conventional urine bag.

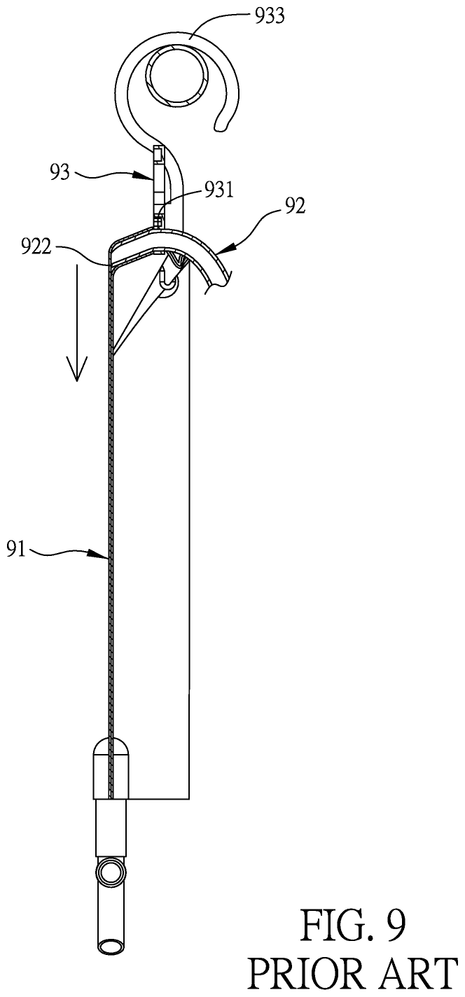

### DESCRIPTION OF THE PREFERRED EMBODIMENTS

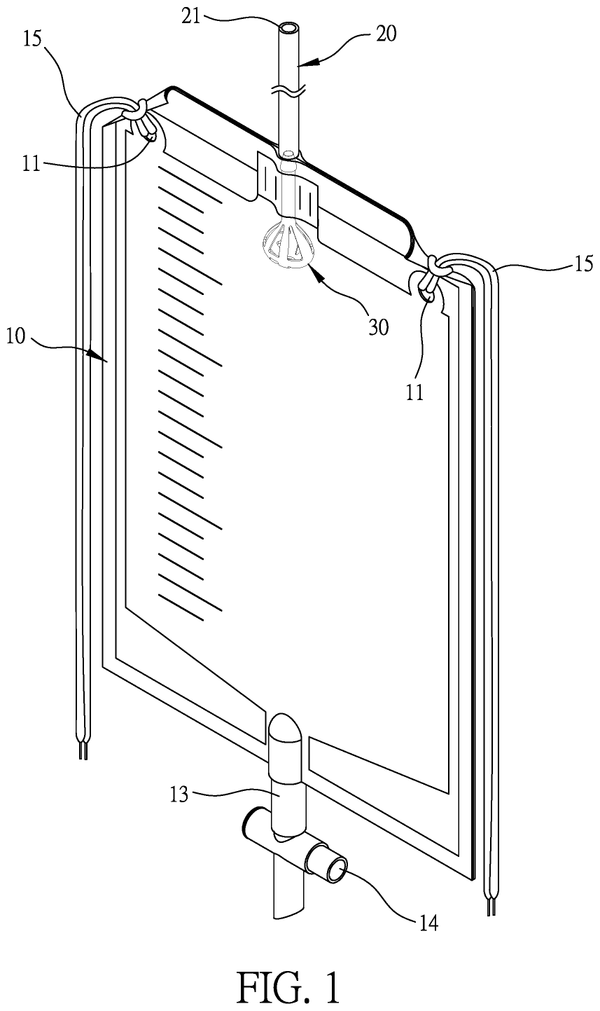

With reference to FIGS. 1 to 4 for an anti-blockage urine bag of this disclosure, the anti-blockage urine bag includes the following elements:

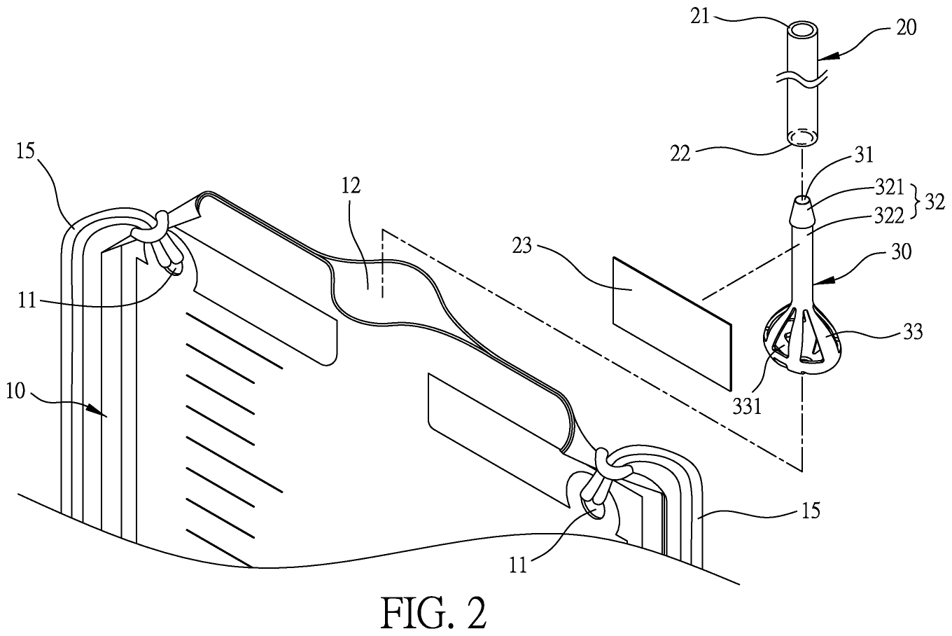

A urine bag body 10 has two fixing holes 11 formed at two corners of the upper side of the urine bag body 10 and a passing hole 12 formed at the middle of the upper side of the urine bag body 10. The passing hole 12 is disposed between the two fixing holes 11 (as shown in FIGS. 1 and 2) and communicated to the interior of the urine bag body 10, and urine can be contained in the urine bag body 10.

A urine drainage tube 13 is connected to the middle of the lower side of the urine bag body 10, the urine drainage tube 13 is installed with a valve device 14 as shown in FIG. 1, the two fixing holes 11 are provided for fixing two positioning strips 15, and the two positioning strips 15 are provided for users to hang the urine bag body 10 at any desired position.

A urethral catheter 20 has a first connection part 21 and a second connection part 22 configured to be opposite to each other, the first connection part 21 is provided for connecting to a user's urinary tract (not shown in the figure), the second connection part 22 is passed through the passing hole 12 of the urine bag body 10 and disposed in the interior of the urine bag body 10, wherein the passing hole 12 of the urine bag body 10 is sealed by a processing method, the processing method can tightly fix the urethral catheter 20 into the sealed passing hole 12 of the urine bag body 10, and the processing method can be a high-frequency welding process or a ultrasonic welding process. In this embodiment of the present disclosure, the processing method is the high-frequency welding process.

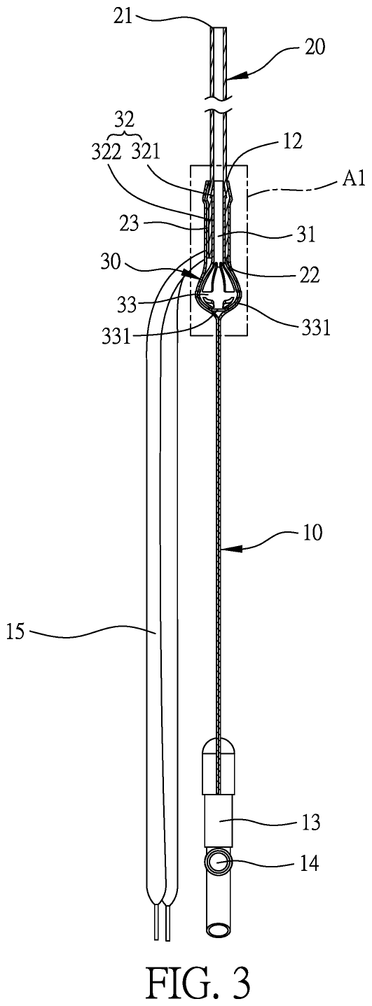

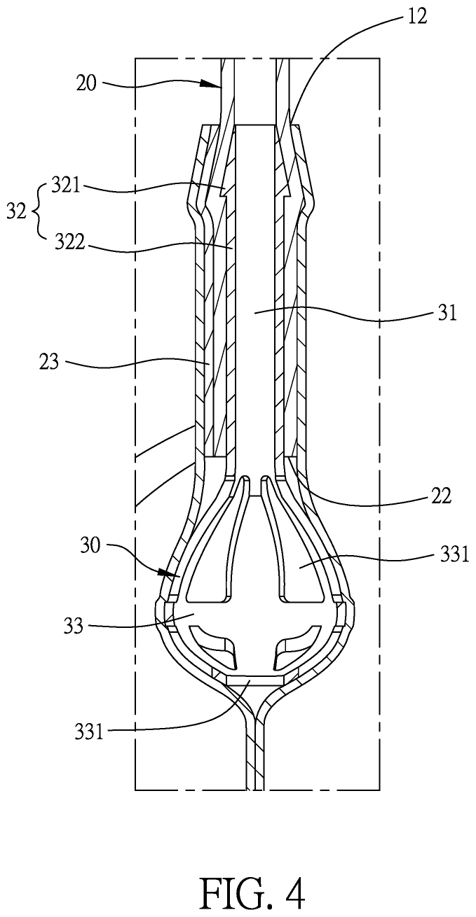

A plastic sheet 23 is disposed between the second connection part 22 of the urethral catheter 20 and the urine bag body 10, and the plastic sheet 23 corresponds to the space between the first connection part 21 and the second connection part 22 covered on the urethral catheter 20 (as shown in FIGS. 3 and 4). The plastic sheet 23 can improve the tightness of sealing the passing hole 12 by the processing method, so that the urine bag body 10 and the urethral catheter 20 can be fixed to each other more tightly to prevent the urine from flowing backward and leaking from the second connection part 22.

An anti-folding tube 30 is communicated between the urine bag body 10 and the urethral catheter 20. The anti-folding tube 30 is made of a hard plastic material and integrally formed. The anti-folding tube 30 has a flow hole 31 penetrating through its interior, and a plug-in part 32 and a hollow support part 33 which are configured to be opposite to each other, and the second connection part 22 of the urethral catheter 20 can be sheathed on the plug-in part 32. The flow hole 31 is communicated to the urethral catheter 20, the plug-in part 32 keeps the urethral catheter 20 straight, and the hollow support part 33 is accommodated in the urine bag body 10 as shown in FIGS. 3 and 4.

The plug-in part 32 has a first portion 321 and a second portion 322, the first portion 321 is in a substantially conical shape, the second portion 322 is in a substantially cylindrical shape, the first portion 321 has a maximum external diameter greater than the external diameter of the second portion 322 and the second portion 322 has a length greater than the length of the first portion 321 as shown in FIGS. 3 and 4. When the urethral catheter 20 is sheathed on the plug-in part 32 of the anti-folding tube 30, the second connection part 22 of the urethral catheter 20 can be quickly sheathed on the minimum external diameter of the first portion 321. As the external diameter of the first portion 321 is expanded to the external diameter of the second portion 322, the urethral catheter 20 can be securely sheathed on the plug-in part 32 to achieve the anti-detachment effect.

Since the external diameter of the hollow support part 33 is greater than the external diameter of the first portion 321 of the plug-in part 32, the hollow support part 33 can hold up the interior of the urine bag body 10. In this embodiment of the present disclosure, the external diameter of the hollow support part 33 is greater than the maximum external diameter of the plug-in part 32.

In addition, the circumferential surface of the hollow support part 33 is substantially ellipsoidal shape and provided with a plurality of skeletonized holes 331, and each skeletonized hole 331 is communicated to the flow hole 31, wherein each skeletonized hole 331 is in a triangular shape, a polygonal shape, or a combination of the above. In this embodiment of the present disclosure, each skeletonized hole 331 is in the combination of a triangular shape and a polygonal shape, but the present disclosure is not limited to such shape, and each skeletonized hole 331 can be of any other geometric shape.

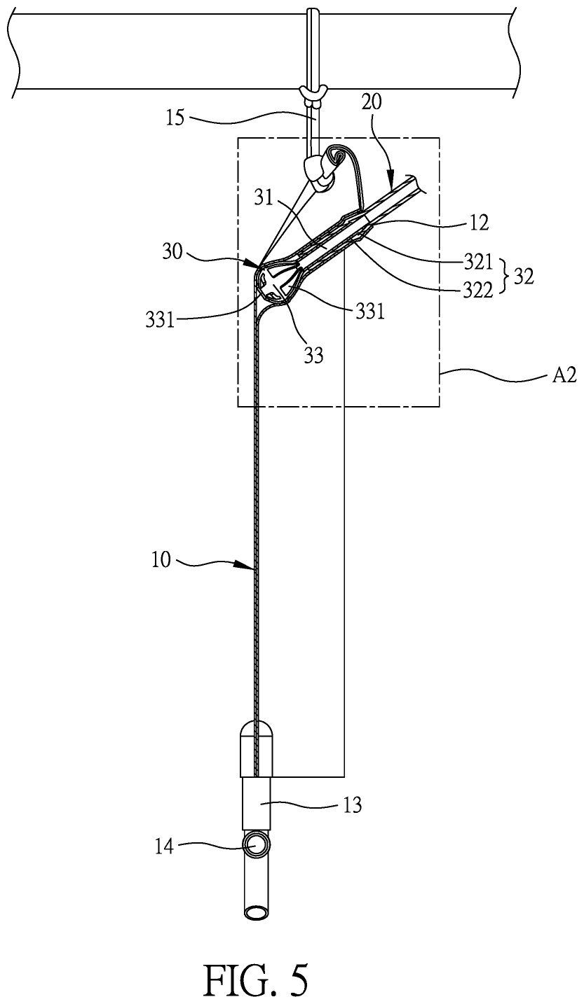

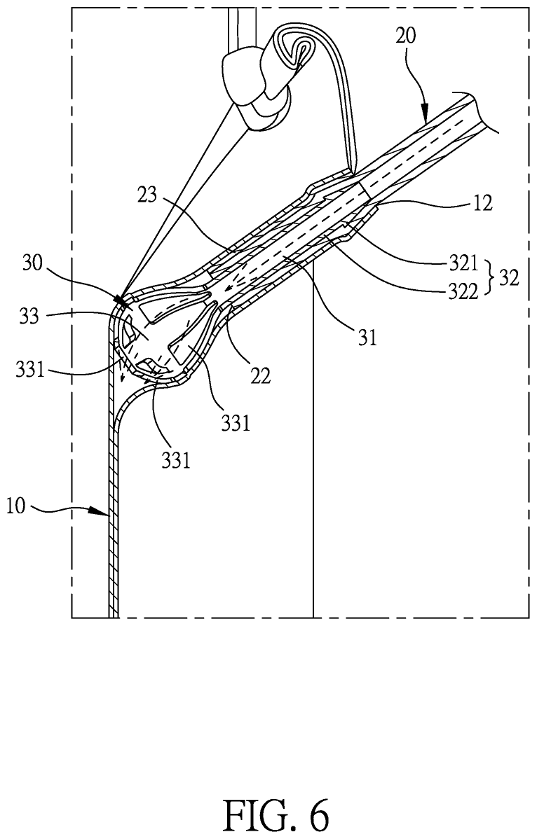

In FIGS. 5 and 6, during use, two positioning strips 15 are tied and fixed to a side of a bed or any other appropriate position, in order to enable the user's urine to flow through the urethral catheter 20 into the urine bag body 10 for collection. When the urethral catheter 20 adjacent to the urine bag body 10 is bent, the plug-in part 32 can keep the urethral catheter 20 straight without affecting the urine bag body 10, and the hollow support part 33 of the anti-folding tube 30 is affixed to the inner wall of the urine bag body 10 to enable the interior of the urine bag body 10 to be held up, so that the urine can flow from the urethral catheter 20 into the flow hole 31 of the anti-folding tube 30 and then to the hollow support part 33. As a result, the urine smoothly flows through each skeletonized hole 331 of the hollow support part 33 into the urine bag body 10 to avoid the blockage of urine.

With the structure of the above exemplary embodiment, the following advantages can be achieved:

1. In the anti-blockage urine bag of the present disclosure, the urethral catheter 20 is passed to an end of the urine bag body 10 which is plugged with the anti-folding tube 30, and the hollow support part 33 of the anti-folding tube 30 is contained in the urine bag body 10, such that the hollow support part 33 can prevent the blockage of urine to ensure the normal smooth catheterization and to enhance practicality.

2. In the anti-blockage urine bag of the present disclosure, the anti-folding tube 30 is integrally formed and plugged into the second connection part 22 of the urethral catheter 20 for connection and conduction, so that the present disclosure has the features of simple structure, convenient assembly, low manufacturing cost, and high cost-effectiveness.

3. In the anti-blockage urine bag of the present disclosure, each skeletonized hole 331 is designed with a non-circular shape, which enables urine to be introduced into the urine bag body 10 more smoothly, so that the urine bag is less likely to be blocked.

What is claimed is:

1. An anti-blockage urine bag, comprising:
a urine bag body, with a passing hole formed on the upper side thereof and communicated to the interior of the urine bag body;
a urethral catheter, coupled and communicated to the urine bag body, and having a first connection part and a second connection part configured to be opposite to each other, the first connection part being provided for connecting to a user's urinary tract, and the second connection part being passed into the passing hole of the urine bag body and disposed inside the urine bag body; and
an anti-folding tube, communicated between the urine bag body and the urethral catheter, and having a plug-in part and a hollow support part, a flow hole being formed and penetrating between the plug-in part and the hollow support part, wherein the second connection part of the urethral catheter is sheathed on the plug-in part, the flow hole is communicated to the urethral catheter, the plug-in part keeps the urethral catheter straight: the hollow support part is contained in the urine bag body, the hollow support part has an external diameter greater than the external diameter of the plug-in part, and the hollow support part is capable of holding up the interior of the urine bag body.

2. The anti-blockage urine bag according to claim 1, wherein the hollow support part is in a substantially ellipsoidal shape, the circumferential surface of the hollow support part is provided with a plurality of skeletonized holes, and each skeletonized hole is communicated to the flow hole.

3. The anti-blockage urine bag according to claim 1, wherein the passing hole of the urine bag body is sealed up by a processing method, and the processing method is capable of fixing the urethral catheter to the urine bag body.

4. The anti-blockage urine bag according to claim 3, wherein the processing method is a high-frequency welding process or an ultrasonic welding process.

5. The anti-blockage urine bag according to claim 1, wherein the urine bag body has two fixing holes penetrating through the upper side thereof, the passing hole is formed between the two fixing holes, and the two fixing holes are provided with two positioning strips respectively.

6. The anti-blockage urine bag according to claim 1, further comprises a urine drainage tube coupled to the middle of the lower side of the urine bag body, and the urine drainage tube is installed with a valve device.

7. The anti-blockage urine bag according to claim 1, further comprises a plastic sheet disposed between the second connection part of the urethral catheter and the urine bag body.

8. The anti-blockage urine bag according to claim 1, wherein the anti-folding tube is made of a plastic material and integrally formed.

9. The anti-blockage urine bag according to claim 1, wherein the plug-in part comprises a first portion and a second portion, the first portion is in a substantially conical shape, the second portion is in a substantially cylindrical shape, and the first portion has a maximum external diameter greater than the external diameter of the second portion.

10. The anti-blockage urine bag according to claim 1, wherein each skeletonized hole is in a triangular shape, a polygonal shape, or a combination thereof.
